(19) United States
(12) Patent Application Publication
(10) Pub. No.: US 2018/0360707 A1
(43) Pub. Date: Dec. 20, 2018

(54) COMPOSITION AND METHOD FOR INCREASE OF SURVIVAL AND STABILIZATION OF PROBIOTIC BACTERIA (PB) IN DETERGENT BASED COMPOSITIONS OF PERSONAL HYGIENE AND DOMESTIC PRODUCTS

(71) Applicant: UAB "PROBIOSANUS", Vilnius (LT)

(72) Inventors: Juozas SIURKUS, Kaisiadorys (LT); Jolanta LIESIENE, Kaunas (LT); Ausra SIPAILIENÈ, Kaunas (LT); Aiste GRUZINSKAITE, Raseiniu r. (LT)

(21) Appl. No.: 16/060,364

(22) PCT Filed: Dec. 31, 2015
(86) PCT No.: PCT/LT2015/000009

§ 371 Date: Jun. 7, 2018

(30) Foreign Application Priority Data
Dec. 10, 2015 (LT) 2015 103

(51) Int. Cl. A61K 8/11; A61K 8/99; A61K 8/73; A61K 8/92; A61K 8/9789; A61K 8/46; A61K 8/41; A61Q 19/10

(52) U.S. Cl. CPC A61K 8/11; A61K 8/99; A61K 8/733; A61K 8/922; A61K 8/9789; A61K 8/463; A61K 2800/596; A61K 8/731; A61Q 19/10; A61K 2800/52; A61K 2800/41; A61K 2800/48; A61K 2800/74; A61K 8/416

(57) ABSTRACT

Disclosed is an approach for probiotic bacteria preservation and protection in the compositions of surface-active material containing personal hygiene/care product compositions. Probiotic bacteria (PB) are well known group of microorganisms for the beneficial effects on human health. Thus it is widely used as food and cosmetic, personal hygiene supplements (C/PHS). PB positive action within the compositions of cosmetics could be related with the complementation of human microbiome, production of various anti-oxidative, moisturizing, biocide activity possessing compounds or other fermentation products which are affecting human skin or other organ systems. In many cases Gram+ PB and/or PB bacterial metabolites are supplemented into detergent based personal care or domestic hygiene compositions to improve product functional features. The PB protection mechanism is based on bacterial immobilization/encapsulation into polymer pectin/alginate or alginate capsules which polymeric barrier allows maintaining of stable bacterial viability in the detergent based personal care or domestic hygiene products.

### FIELD OF INVENTION

Present invention relates to a novel approach for preservation of probiotic bacteria by encapsulation in polymeric particles with the aim to protect them from surfactants and other deteriorating compounds which are commonly used in personal care products including their final formulations.

### BACKGROUND

Probiotic bacteria are a group of non-pathogenic microorganisms, which are beneficial to human and animal health in various different mechanisms. The probiotic action could be related with the following mechanisms: (i) secretion of bactericides—metabolites, peptides and enzymes, pH modulation, biocompetition (ii), biofilm or barrier formation and immunomodulation (1—Walker W A. (2008) Mechanisms of action of probiotics. Clin Infect Dis. 46 (Supplement 2): S87-S91). Probiotics may alter skin health and, for example, might be effective at preventing the development of atopic dermatitis (2—Yeşilova, Y., Çalka, Ö., Akdeniz, N., & Berktaş, M. (2012). Effect of Probiotics on the Treatment of Children with Atopic Dermatitis. Annals of Dermatology, 24(2), 189-193), including prevention of skin diseases and improvement of skin condition (3—Reid, G., Jass, J., Sebulsky, M. T., & McCormick, J. K. (2003). Potential Uses of Probiotics in Clinical Practice. Clinical Microbiology Reviews, 16(4), 658-672.). Some bacterial metabolites might act as antioxidants and thus reduces or prevents melanogenesis or other ROS including chemical agent related skin damage (4—Tsao, R., Yang, R. and Young, J. C. (2003) Antioxidant isoflavones in orange, maclura pomifera (Raf.) Scheneid. J Agric Food Chem 51, 6445-6451, 5—Wang, Y. C., Yu, R. C. and Chou, C-C. (2006) Antioxidative activities of soymilk fermented with lactic acid bacteria and bifidobacteria. Food Microbiol 23, 128-135). The health beneficial features of the probiotic or/and their metabolites for the skin is one of the factors which made them to be used as an active compounds in the compositions of personal hygiene or cosmetic products. The aim of probiotic bacteria within the cosmetic products is to diminish indications related with skin infections, pustules, skin fat balancing and reduction of dermatitis. One of examples is the patent EP 2306970 A1, which covers the invention of cosmetic compositions comprising lacto and/or bifido bacteria compounds in the range of 0.001-20% (w/w) in combination with vitamins (B3, B5, B6, C, D, E, PP) niacine, carotenoids, polyphenols and minerals (Zn, Ca, Mg, Cu, Fe, I, Mn, Se, Cr), phytoestrogens, proteins, amino acids, mono- and polysaccharides, phytoterpenes, phytosterols and aminosugars. The Lactobacillus paracasei ST1 was used as the dominating bacterial in the described compositions. The patent application also describes the formats of probiotic based cremes, gels, powder and tablets (6—Patent application EP 2306970 A1).

Patent application FR 2889057 describes a cosmetic composition comprising lactic acid bacteria with unsaturated fatty acids and their esters (7—Patent application FR 2889057). Similarly, patent application WO 2006/07922 describes a cosmetic composition for sensitive skin, which is based on Lactobacillus paracasei or casei and Bifidobacterium longum or Bifidobacterium lactis bacteria in combination with other active ingredients (8—WO 2006/07922). Applications WO 02/28402 and WO 03/070260 describes microbial based cosmetic compositions for sensitive skin with calming features and for protection of skin from damaging UV effects, respectively (9 patent application WO 02/28402, 10 and patent application WO 03/070260).

None of these IP protected compositions are referring to the probiotic or probiotic-based metabolite stability aspects in the direct contact with the chemical compounds of cosmetic products. The probiotic bacterial stability in various composition issues could be related with bactericide, bacteriostatic and lytic effects of cosmetic composition compounds especially surface active materials, preservatives, terpenes, alcohols and other. The surface active materials (SAMs) are the key compounds in the cosmetic and personal hygiene products, which could be for the functions of cleaning, moisturising, foaming or antifoaming and distributing/spreading and emulsifying. SAMs are water-soluble polymers due to so called amphophilic feature, where a molecule is composed of hydrophobic tale and hydrophilic moiety. In the water solutions SAMs are forming micelles—structured units due to the hydrophobic tail, which is excluding polar phase and hydrophilic residue which is oriented to the solute. According to the features of the molecules the SAMs are subdivided into anionic, cationic and non-polar (11—Surface-Active Agents: Advances in Research and Application: 2011 Edition. Edited by Ashton Acto). The most powerful cell lysis potential of bacterial cells are possessing the strong, polar SAMs which action of mechanism is based the interaction with bacterial membrane hydrophobic and hydrophilic patches, respectively. As the result of this interaction the bacterial cell membranes are ruptured resulting in the cell lysis and release of intracellular content—soluble proteins, ribosomes, DNA and RNA, into the environment (12—Vasily N. Danilevich, Lada E. Petrovskaya, Eugene V. Grishin (2008) A Highly Efficient Procedure for the Extraction of Soluble Proteins from Bacterial Cells with Mild Chaotropic Solutions. Chem. Eng. Technol. 31, 6: 904-910). The bacterial stability against various physical and chemical factors can be increased by encapsulation into the various polymer capsules or immobilization into surfaces of various solid particles (13—Riaz Q U, Masud T. Recent trends and applications of encapsulating materials for probiotic stability. (2013) Crit Rev Food Sci Nutr. 53(3):231-44). There are several technologies, which are used for bacterial encapsulation: spray drying, extrusion and emulsification. The formed particles can be further stabilized by lyophilisation. The materials, which could be used for bacterial encapsulation are polysaccharides for example alginate, polysaccharides and lipids, and proteins. Depending on the encapsulation methodology and materials bacterial several main types of polymer capsules could be obtained—reservoir (bacterial cells are in the suspension, inside the capsule cavity) embedded inside the capsule polymer matrix and coated with polymer matrix. In addition, combinations of different polymers and encapsulation methodologies allow varying capsule sizes in range of 3 μm-5 mm. Finally the bacterial cells release from capsules could be obtained by temperature treatment, moisture change, pH shifts, enzymatic treatment and exposure with mechanical force. The aim of excising bacterial encapsulation approaches is to protect probiotic bacterial cells in the digestion track of the human and animals against various hush factors conditions—usually extremely low pH (13—Riaz Q U, Masud T. Recent trends and applications of encapsulating materials for probiotic stability. (2013) Crit Rev Food Sci Nutr. 53(3):231-44).

### SUMMARY OF INVENTION

Probiotic bacteria (PB) are well known group of microorganisms for the beneficial effects on human health. Thus it is widely used as food and cosmetic, personal hygiene supplements (C/PHS).

PB positive action within the compositions of cosmetics could be related with the complementation of human microbiome, production of various anti-oxidative, moisturizing, biocide activity possessing compounds or other fermentation products which are directly or indirectly affecting human skin or other organ systems.

In many cases Gram+ PB and/or PB bacterial metabolites are supplemented into detergent based personal care or domestic hygiene compositions with the aim to improve product functional features. However, the key compounds of (C/PHS): surface active materials (detergents: cationic—Sodium laureth sulfate (SLS), Sodium Cocoyl Glutamate, amphoteric: benzalkonium chloride, Cetrimonium chloride, Cocamidopropyl betaine, cocoamphoacetate, sodium cocoyl glycinate, non-ionic: diethanolamine and Coco-glucoside), conserving materials, terpenes, are directly acting on the PB as biocides, thus over the incubation time in these compositions, e.g. during product storage and usage the probiotic bacteria becoming inactivated—killed, resulting in the loss of the product composition and function. In addition, the metabolites of PB bacteria could interact with product compounds, could be exposed to UV light and/or oxidized and therefore chemically altered and/or inactivated.

We have used and approach for protection of probiotic bacteria, against the compounds of detergent based personal care or domestic hygiene products comprising bactericidal surface active materials—cationic, amphoteric and non-ionic detergents, preserving materials, terpenes (fragrances) and complex compositions comprising materials of groups mentioned above. The PB protection mechanism is based on bacterial immobilization/encapsulation into polymer pectin/alginate or alginate capsules which polymeric barrier allows maintaining of stable bacterial viability in the detergent based personal care or domestic hygiene products.

The PB protection mechanism is based on bacterial immobilization/encapsulation into polymer such as polysaccharides (e.g., pectin/alginate or alginate) or/and polysaccharides and lipids, or/and proteins capsules which polymeric barrier allows maintaining of stable bacterial viability in the detergent based personal care or domestic hygiene products.

As the example we demonstrated this invention using Bacillus coagulans bacteria and its metabolic compounds, by incubating known titters of encapsulated and non capsulated bacterial cells in various detergent compositions, personal care products.

We have experimentally showed that polymeric encapsulation results in the stabilization of alive PB titters, or decreases the bacterial inactivation during the 120 hours at the elevated temperatures −37° C., (which could be an equivalent to 120 days) of encap-bacterial incubation in the various detergent compositions within the personal hygiene products. At these experimental conditions we observed that viable encap-bacterial preservation is corresponding to survival of 60-90% compared to the analogical compositions with non-encapsulated bacteria, where 60-90% of added bacteria to the composition were inactivated during the same incubation conditions.

### DESCRIPTION OF FIGURES

FIG. 1. Provides results of survival of encapsulated and non-encapsulated B. coagulans bacteria after incubation in the solution comprising amphoteric surface active material—Cocoamido Propyl Betain.

FIG. 2. Provides results of survival of encapsulated and non-encapsulated B. coagulans bacteria after incubation in the solutions comprising non-ionic surface active materials—2%, or Cocoglycozide-methyl oleate 3%, or Cocamide Diethanolamine or Cocoglycozide.

FIG. 3. Provides results of survival of encapsulated and non-encapsulated B. coagulans bacteria after incubation in the solutions of Grapefruit seed extract, or d-limonene, or Azadirachta indica extract.

FIG. 4 Provides survival results of B. coagulans bacteria in the polymer-encapsulated system and non-capsulated B. coagulans bacteria after incubation in the solution of non-ionic surface active materials comprising MILD HANDS WASHING-UP LIQUID WITH PROVITAMIN B5 (UAB Probiosanus) or PET SHAMPOO FOR LONG COAT B5 (UAB Probiosanus) or PET SHAMPOO FOR SHORT COAT (UAB Probiosanus).

### MATERIALS AND METHODS

Bacterial Cell Preparation

For all experimental trails probiotic strain of Bacillus coagulans DSM 2311 was used which was obtained from DSMZ. Bacterial cell suspension was prepared by cultivation of B. coagulans cells in 100 mL of liquid nutrient broth medium (Oxoid) in 1000 mL Erlenmeyer flask, for 24 hours at 37° C. while shaking at 180 rmp. Prior to encapsulation the cells were collected by centrifugation at 6000 rpm using Rotofix 32 A Hettich, then washed twice and re-suspended in sterile 0.9% NaCl solution to obtain cell concentration of 10¹¹ per mL.

Bacterial Cell Encapsulation

Encapsulation of B. coagulans cells was performed using emulsion formation approach. 200 mL of emulsion comprising of 40% water phase and 60% sunflower-seed oil was prepared and mixed to obtain homogenous consistency.

Water phase was composed of 2.4% alginate and B. coagulans bacterial cells to obtain 10⁹-10¹⁰ of bacterial cells per gram of capsules. Oil phase was composed of 99.5% sunflower-seed oil with 1% of POLYSORBATE 80. To obtain the emulsion, bacteria-alginate water phase was slowly introduced by pouring it into the oil phase solution and mixed for at least 20 minutes at room temperature. After emulsion was formed, sterile 5.5% CaCl₂ solution was slowly added to obtain final concentration of 3%. The reaction mixture was then incubated at the room temperature for several hours. During incubation the solution was exposed to a UV light source to reduce possibility of microbial contamination. The obtained bacterial-cell-harbouring capsules were collected by centrifugation at low G and washed twice with sterile deionised water and stored in 0.1% tryptone water at +4° C. The bacterial titre per mass of the capsules was obtained 10¹⁰/g of wet weight.

Optionally, for further experimentation, capsules were lyophilised for 24 h at −110° C. in Christ ALPHA 2-4 LSC and stored at −20° C. or −80° C. The size of the matured capsules was determined using Malvern Mastersizer 2000 and varied in the range of 0.2-0.24 mm lyophilized (30 minutes after rehydration in water) and 0.2-0.260 mm non-lyophilized, respectively.

Analysis of Encapsulated Bacteria Viability in Detergent Based Solution.

1 g of ˜10¹⁰ bacteria-containing lyophilized and rehydrated or fresh microcapsules was added to the fixed volume (100 mL) of either: detergent solution, cosmetic compound containing solution or final formulation detergent based product, and incubated at 37° C. temperature. The samples were harvested at certain timepoints to evaluate bacterial survival. After sample harvest the bacterial capsules were washed twice with sterile water and added to a fixed volume of sterile 2% sodium citrate solution for liberation of encapsulated bacteria. The cell suspension was diluted from 10¹ to 10⁵ fold and 100 μL of solution was plated on Petri dishes filled with Nutrient Broth solid medium. The survival rates [%] of capsulated and non-capsulated bacteria was estimated by comparing CFU averages at each sample point, from 3 replicas. See formulas:

$$N ( CFU ) = ( \frac{CFU * Vfd}{Vp} ) * D$$

N—estimated CFU;
CFU—sum of calculated colonies from each evaluated Petri plates
Vₚ—plated volume
Vfd—final dilution volume
D—dilution fold
Survival of bacteria [%]=N₍₁₊ₙ₎/N₁*100
N₁—bacterial CFU's from first timepoint sample
N₍₁₊ₙ₎—bacterial CFU's from following timepoint sample

### EMBODIMENTS OF INVENTION

### Example 1

1 g of 10¹⁰ bacteria-containing rehydrated or fresh microcapsules was added to the fixed volume (100 mL) of detergent solution and incubated for 120 hours at 37° C. temperature. A parallel experiment was performed with non-capsulated cells (control group). After the sample harvest the bacterial capsules were washed twice with sterile water and added to the fixed volume of sterile 2% sodium citrate solution for liberation of encapsulated bacteria. The cell suspension was diluted 10¹-10⁵ fold and 100 μL plated on replicates of Nutrient Broth solid medium. The survival rates [%] of capsulated and non-capsulated bacteria were estimated as described in the section—Analysis of encapsulated bacteria viability in detergent based solution, by comparing CFU averages at each sample time point from 3 replicas.

a. Non-capsulated and encapsulated bacteria survival in solution of amphoteric surface-active material—cocoamide propyl betaine (CAPB) 6.7% (stock concentration 99%, BASF).

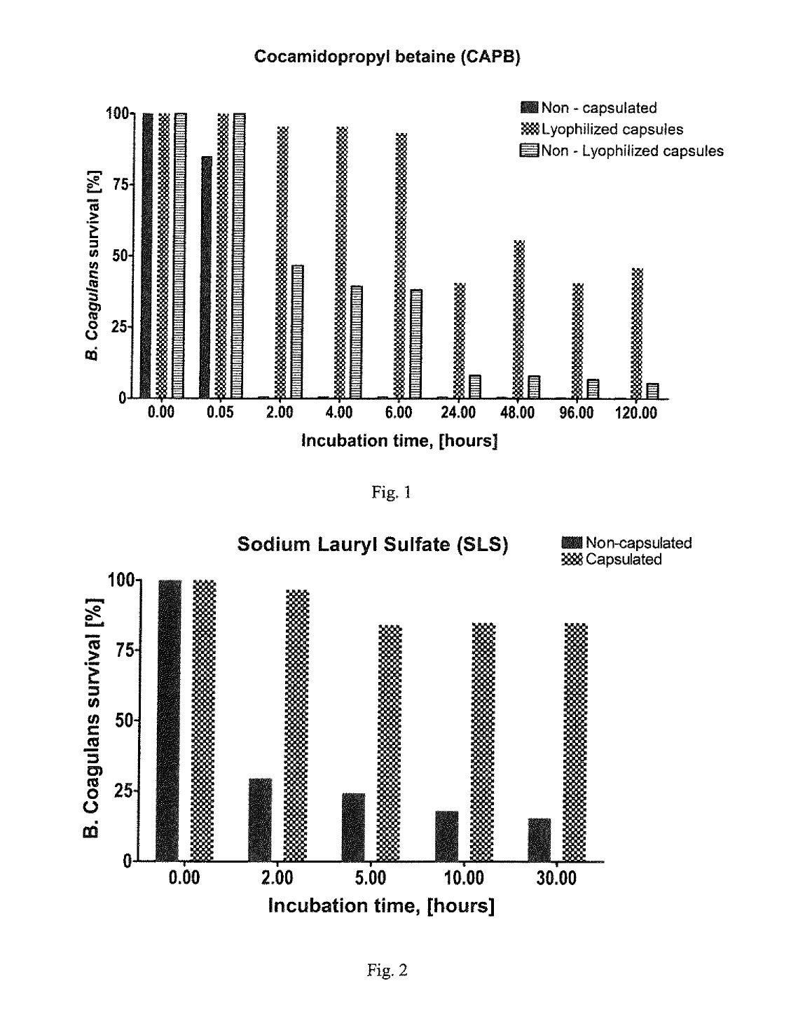

The experiments revealed that 97% of non-capsulated bacteria (starting titers) were inactivated after 0.5 h due to the exposure to the CAPB, while ˜50% and ˜35% survived in lyophilized and non-lyophilized capsules after incubation for 120 h at 37° C., respectively (FIG. 1).

Conclusions: encapsulation effectively preserved B. coagulans bacteria in solution of amphoteric surface active material—cocoamido propyl betain while this compound inactivated the same strain of non-capsulated bacteria.

b. Non-capsulated and lyophilized encapsulated bacteria survival in anionic surface active material—6.7% sodium lauryl sulphate (SLS) (stock concentration 98%, BASF) solution.

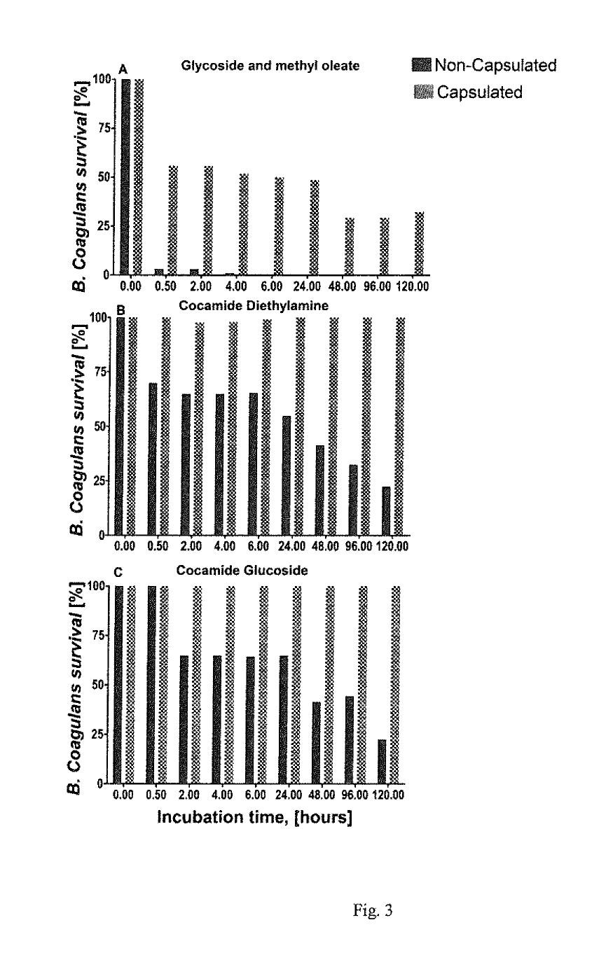

The experiments revealed that 80% of non-capsulated bacteria were inactivated due to exposure to SLS, while ˜80% survived in lyophilized capsules after incubation for 120 h at 37° C. (FIG. 2).

Encapsulation effectively preserved B. coagulans bacteria in SLS solution while this compound inactivated the same strain of non-capsulated bacteria.

c. Non-capsulated and encapsulated lyophilized bacteria survival in non-ionic surface active materials: 2% cocoglycozide-methyl oleate (stock concentration 98%, BASF), 3%, cocamide diethanolamine (stock concentration 98%, BASF) and 7% cocoglycozide (stock concentration 98% BASF) (Panel C) solutions.

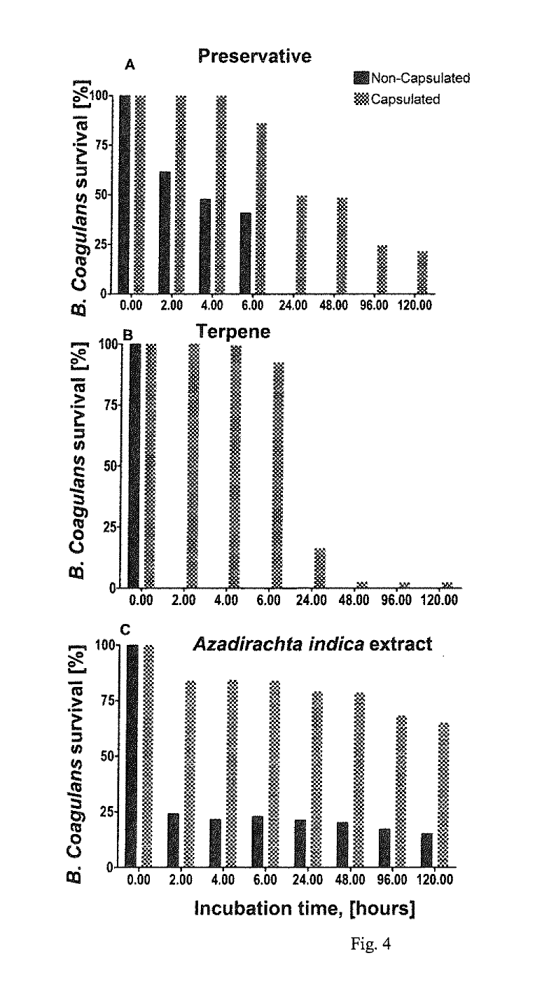

The experiments revealed that 98% of non-capsulated bacteria were inactivated due to the exposure to cocoglycozide-methyl oleate after 0.5 h incubation, while ˜50% survived for 120 h in lyophilized capsules (Panel A); 75% of non-capsulated bacteria were inactivated due to the exposure to cocamide diethanolamine (Panel B) and cocoglycozide (Panel C), while >95% survived in lyophilized capsules after incubation for 120 h at 37° C. (Panel B and C) FIG. 3.

Encapsulation effectively preserved B. coagulans bacteria in compositions comprising natural non-ionic surface active materials while these compounds inactivated the same strain of non-capsulated bacteria.

### Example 2

1 g of 10¹⁰ bacteria containing lyophilized-rehydrated microcapsules was added to the fixed volume (100 mL) of solutions of: Grapefruit seed extract d-limonene, or Azadirachta indica extract and incubated for 120 hours at 37° C. temperature. A parallel experiment was performed with non-capsulated cells. After sample harvest the bacterial capsules were washed twice with sterile water and added to the fixed volume of sterile 2% sodium citrate solution for liberation of encapsulated bacteria. The cell suspension was diluted 10¹-10⁵ fold and 100 μL of solution was plated on replicates of Nutrient Broth solid medium. The survival rates [%] of capsulated and non-capsulated bacteria were estimated as described in the section—Analysis of encapsulated bacteria viability in detergent based solution, by comparing CFU averages at each sample point, from 3 replicas.

a. Non-capsulated and encapsulated lyophilized bacteria survival in solution of 0.6% preservative—Grapefruit seed extract ((Citrus paradisi)—stock concentration 98%, LemonConcentrate) (panel A)), 0.7% terpene, d-limonene (stock concentration 99%, LemonConcentrate) (panel B) or 5% Azadirachta indica extract (stock concentration, 90%, JSC “Baltkos”) (Panel C).

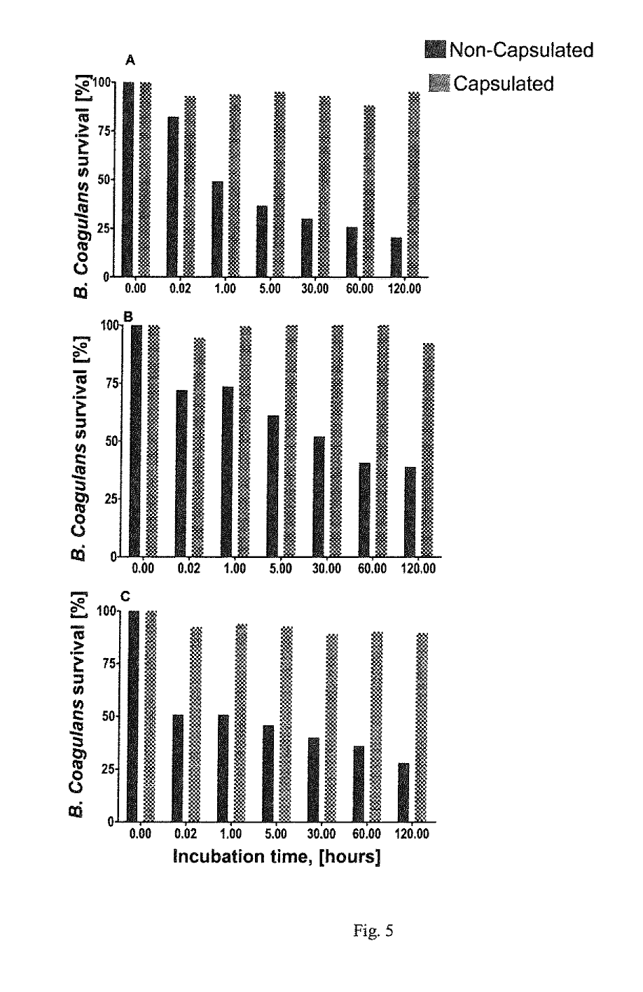

The experiments revealed that 98% of non-capsulated B. Coagulans bacteria were inactivated due to exposure to the preservative—Grapefruit seed extract after 6 h of incubation inactivated instigated B. Coagulans bacteria >90%, while >50% survived for 24 h and >35% after 120 h in lyophilized capsules (Panel A); 100% of non-capsulated B. Coagulans bacteria were inactivated in 2 hours due to the exposure to terpenes (Panel B) and 75% due to the action of Azadirachta indica extract (Panel C), while ˜80% of B. Coagulans survived in lyophilized capsules after incubation at 37° C. for 6 h and 75% for 120 h, respectively (FIG. 4 (Panel B and C)).

Encapsulation effectively preserved B. coagulans bacteria in compositions comprising natural seed-extract based preservative, terpenes and Azadirachta indica extract while these compounds inactivated the same strain of non-capsulated bacteria.

### Example 3

In this experiment, as detergent containing formulations were used three commercial products, all of UAB Probiosanus: MILD LIQUID HAND WASH WITH PROVITAMIN B5 with the composition listed in the Table 1; PET SHAMPOO FOR LONG COAT B5 with the composition listed in Table 2 and PET SHAMPOO FOR SHORT COAT UAB Probiosanus) with the composition listed in Table 3.

1 g of 10¹⁰ bacteria containing lyophilized—rehydrated microcapsules was added to the fixed volume (100 mL) of detergent containing formulation and incubated for 120 hours at 37° C. temperature. A parallel experiment was performed with non-capsulated cells. After sample harvest the bacterial capsules were washed twice with sterile water and added to the fixed volume of sterile 2% sodium citrate solution for liberation of encapsulated bacteria. The cell suspension was diluted 10¹-10⁵ fold and 100 μL of solution was plated on replicates of Nutrient Broth solid medium. The survival rates [%] of capsulated and non-capsulated bacteria were estimated as described in the section—Analysis of encapsulated bacteria viability in detergent based solution, by comparing CFU averages at each sample point, from 3 replicas.

Results.

Non-capsulated and encapsulated bacteria survival in lyophilized alginate micro capsules (diameter −0.24 mm) in solution of non-ionic surface active materials comprising MILD LIQUID HAND WASH WITH PROVITAMIN B5.

TABLE 1
MILD LIQUID HAND WASH WITH PROVITAMIN
B5 (UAB Probiosanus)

| | | Mass, |
|---|---|---|
| Compound | CAS Nb. | [%] |
| Sodium laureth sulfate (SLS | 68891-38-3 | 6.4 |
| concentration 70%) | | |
| Alkyl (C8-C10) polyglycozide | 68515-73-1 | 3.6 |
| (concentration 70%) | | |
| (Cocamide DEA, concentration | 68603-42-9 | 2.8 |
| 85%) | | |
| Glycerol | 56-81-5 | 4 |
| Orange terpenes | 5989-27-5 | 0.35 |
| Fragrance | 233799 | 0.1 |
| D-pantenol | — | 0.4 |
| Ethyl hydroxyethyl cellulose | 9004-58-4 | 0.9 |
| (EHEC) | | |
| Preservative (Orange terpenes) | 26172-55-4 | 0.2 |
| | 2682-20-4 | |
| | 100-51-6 | |
| Citric acid | 5949-29-1 | 0.015 |

TABLE 2
PET SHAMPOO FOR LONG COAT B5 (UAB Probiosanus)

| | | Mass, |
|---|---|---|
| Compound | CAS Nb. | [%] |
| Sodium Cocoyl Glutamate | 68187-30-4 | 10 |
| (concentration 99%) | | |
| Coco Glucoside (concentration | 110615-47-9, | 7.0 |
| 99%) | 68515-73-1 | |
| Coco Betaine (concentration 99%) | 66455-29-6 | 5.0 |
| Neem extract (Azadirachta indica) | 84696-25-3/ | 1 |
| | 90063-92-6 | |
| Chamomila recutita extract | — | 1 |
| Nettle extract | 84012-40-8 | 1 |
| Xanthan gum | 11138-66-2 | 0.75 |
| Lactic acid/80 | 598-82-3 | 0.5 |
| Preservative ACNIBIO C PLUS | 90045-43-5 | 0.06 |

TABLE 3
PET SHAMPOO FOR SHORT COAT B5 (UAB Probiosanus)

| | | Mass, |
|---|---|---|
| Compound | CAS Nb. | [%] |
| Caprylyl Capryl Glucoside | 68515-73-1 | 5.38 |
| (concentration 99%) | | |
| Sodium Cocoyl Glutamate | 68187-30-4 | 4 |
| (concentration 99%) | | |
| Coco Betaine (concentration 99%) | 66455-29-6 | 6.7 |
| Glyceryl Oleate (and) Coco | 77-92-9, | 3,.85 |
| Glucoside | 141464-42-8 | |
| Neem extract (Azadirachta indica) | 84696-25-3/ | 1 |
| | 90063-92-6 | |
| Chamomila recutita extract | — | 1 |
| Nettle (Urtica dioica) extract | 84012-40-8 | 1 |
| Xanthan gum | 11138-66-2 | 0.75 |
| Preservative - citrus seed extract | 90045-43-5 | 0.06 |

The experiments revealed that 80% of non-capsulated bacteria were inactivated after incubation for 120 h at 37° C. due to the exposure to MILD LIQUID HAND WASH WITH PROVITAMIN B5 comprising major bacteriocidic components (i) SAMs: Sodium laureth sulfate, Alkyl (C8-C10) polyglycozide and Cocamide DEA (ii) terpenes: Orange terpenes, while >95% survived in lyophilized capsules (Panel A); 60% of non-capsulated bacteria were inactivated after incubation for 120 h at 37° C. due to the exposure to PET SHAMPOO FOR LONG COAT B5 comprising (i) SAMs: Sodium Cocoyl Glutamate, Coco Glucoside, Coco betain (ii) Plant extracts: Neem extract (Azadirachta indica), Nettle (Urtica Dioica) and (iii) Preservative: ACNIBIO C PLUS which is based on ascorbic acid (vitamin C) and bioflavonoids from citric fruit (vitamin P): (Panel B) and ˜75% PET SHAMPOO FOR SHORT COAT (Panel C) comprising (i) SAMs: Sodium Cocoyl Glutamate, Coco Glucoside, Coco betain and: Glyceryl Oleate (and) Coco Glucoside, (ii) Plant extracts Neem extract (Azadirachta indica) and Chamomila recutita, Nettle (Urtica dioica), (iii) Preservative based on citrus seed extract, while ˜>90% of B. Coagulans survived in lyophilized capsules after incubation in the both for 120 h at 37° C. (FIG. 5 (Panel B and C)).

Conclusions: encapsulation effectively preserved B. coagulans bacteria in the personal hygiene/care product compositions comprising combinations of anionic and/or non-ionic surface and/or amphoteric surface active materials with natural seed-extracts and preservative based on terpenes and vitamins, while the same compositions inactivated non-capsulated B. coagulans bacteria.

### LITERATURE AS CITED

1. Walker W A. (2008) Mechanisms of action of probiotics. Clin Infect Dis. 46 (Supplement 2): S87-S91
2. Yeşilova, Y., Çalka, Ö., Akdeniz, N., & Berktaş, M. (2012). Effect of Probiotics on the Treatment of Children with Atopic Dermatitis. Annals of Dermatology, 24(2), 189-193.
3. Reid, G., Jass, J., Sebulsky, M. T., & McCormick, J. K. (2003). Potential Uses of Probiotics in Clinical Practice. Clinical Microbiology Reviews, 16(4), 658-672.
4. Tsao, R., Yang, R. and Young, J. C. (2003) Antioxidant isoflavones in orange, maclura pomifera (Raf.) Scheneid. J Agric Food Chem 51, 6445-6451.
5. Wang, Y. C., Yu, R. C. and Chou, C-C. (2006) Antioxidative activities of soymilk fermented with lactic acid bacteria and bifidobacteria. Food Microbiol 23, 128-135.
6. Patent application EP 2306970 A1
7. Patent application FR 2889057
8. Patent application WO 2006/07922
9. Patent application WO 02/28402
10. Patent application WO 03/070260
11. Surface-Active Agents: Advances in Research and Application: 2011 Edition. Edited by Ashton Acton
12. Vasily N. Danilevich, Lada E. Petrovskaya, Eugene V. Grishin (2008) A Highly Efficient Procedure for the Extraction of Soluble Proteins from Bacterial Cells with Mild Chaotropic Solutions. Chem. Eng. Technol. 31, 6: 904-910
13. Riaz Q U, Masud T. Recent trends and applications of encapsulating materials for probiotic stability. (2013) Crit Rev Food Sci Nutr. 53(3):231-44.
14. Patent aplication WO2015000972A1
15. Patent aplication WO2015019307A1
16. Patent aplication US20080107699A1
17. Patent aplication WO2004035885A1
18. WO2013188626A2
19. US20140065218A1
20. U.S. Pat. No. 9,157,054B2
21. WO2010045541A1
22. US20150071977A1

1. Liquid composition comprising:
polymeric capsules with encapsulated viable Gram positive or/and viable Gram negative bacterial cells, wherein said polymeric capsules comprises polysaccharides or/and polysaccharides and lipids, or/and proteins; and
at least one bactericidal surface active material or combination thereof, wherein the bactericidal surface active materials are selected from ionic or/and non-ionic, or/and amphoteric; and
at least one of phyto-extracts, or/and terpenes or/and alcohols, or/and fatty acids, or/and metabolites, or/and organic acids, and/or vitamins, and/or bacteriocidic/static preservative, and fragrances, and thickeners.

2. Liquid composition of claim 1, wherein polymeric capsules size ranges from 0.2 mm to 5 mm.

3. Liquid composition according to claim 1, wherein encapsulated viable Gram positive or/and viable Gram negative bacterial cells count is from 1 to 1*10⁹ per polymer particle, from 1 to 1*10¹² of viable cells per gram of capsules wet weight.

4. Liquid composition according to claim 1, wherein said thickeners are selected from Xanthan gum and Celluloses in combination or separately.

5. Liquid composition according to claim 1, wherein said polymer comprises alginate.

6. Liquid composition according to claim 1, wherein said at least one bactericidal surface active material is cocoglycozide-methyl oleate or/and cocamide diethanolamine or/and cocoglycozide.

7. Liquid composition according to claim 1, wherein said at least one bactericidal surface active material is cocoamide propyl betaine.

8. Liquid composition according to claim 1, wherein said at last one bactericidal surface active material is sodium lauryl sulphate.

9. Liquid composition according to claim 1, said preservative is grapefruit seed extract or/and terpene or/and d-limonene.

10. Liquid composition according to claim 1, wherein said fragrancies are selected from plant fruiting body, fruit or seed extract/oil form Azadirachta indica, Urtica Dioica, Chamomila recutita, Cannabis sativa extract.

11. Liquid composition according to claim 1, wherein the composition consists of 1 g of polymeric capsules with encapsulated viable bacterial cells in admixture with 100 ml of combination of bactericidal surface active materials the components ratio of the latter being as follows:

| | | Mass, |
|---|---|---|
| Compound | CAS Nb. | [%] |
| Sodium laureth | 68891-38-3 | 6.4 |
| sulfate (SLS 70%) | | |
| Alkyl (C8-C10) | 68515-73-1 | 3.6 |
| polyglycozide (70%) | | |
| (Cocamide DEA 85%) | 68603-42-9 | 2.8 |
| Glycerol | 56-81-5 | 4 |
| Orange terpenes | 5989-27-5 | 0.35 |
| Fragrance | 233799 | 0.1 |
| D-pantenol | — | 0.4 |
| Ethyl hydroxyethyl | 9004-58-4 | 0.9 |
| cellulose (EHEC) | | |
| Preservative (Orange | 26172-55-4 | 0.2 |
| terpenes) | 2682-20-4 | |
| | 100-51-6 | |
| Citric acid | 5949-29-1 | 0.015 |

12. Liquid composition according to claim 1, wherein the composition consists of 1 g of polymeric capsules with encapsulated viable bacterial cells in admixture with 100 ml of combination of bactericidal surface active materials the components ratio of the latter being as follows:

| | | Mass, |
|---|---|---|
| Compound | CAS Nb. | [%] |
| Sodium Cocoyl | 68187-30-4 | 10 |
| Glutamate | | |
| Coco Glucoside | 110615-47-9, | 7.0 |
| | 68515-73-1 | |
| Coco Betaine | 66455-29-6 | 5.0 |
| Neem extract | 84696-25-3/ | 1 |
| (Azadirachta indica) | 90063-92-6 | |
| Chamomila recutita | — | 1 |
| extract | | |
| Nettle extract | 84012-40-8 | 1 |
| Xanthan gum | 11138-66-2 | 0.75 |
| Lactic acid/80 | 598-82-3 | 0.5 |
| Preservative ACNIBIO C | 90045-43-5 | 0.06 |
| PLUS | | |

13. Liquid composition according to claim 1, wherein the composition consists of 1 g of polymeric capsules with encapsulated viable bacterial cells in admixture with 100 ml of combination of bactericidal surface active materials the components ratio of the latter being as follows:

| | | Mass, |
|---|---|---|
| Compound | CAS Nb. | [%] |
| Caprylyl Capryl Glucoside | 68515-73-1 | 5.38 |
| Sodium Cocoyl Glutamate | 68187-30-4 | 4 |
| Coco Betaine | 66455-29-6 | 6.7 |
| Glyceryl Oleate (and) | 77-92-9, | 3,.85 |
| Coco Glucoside | 141464-42-8 | |
| Neem extract (Azadirachta | 84696-25-3/ | 1 |
| indica) | 90063-92-6 | |
| Chamomila recutita | — | 1 |
| extract | | |
| Nettle (Urtica dioica) | 84012-40-8 | 1 |
| extract | | |
| Xanthan gum | 11138-66-2 | 0.75 |
| Preservative - citrus | 90045-43-5 | 0.06 |
| seed extract | | |

14. Encapsulated viable Gram positive or/and viable Gram negative bacterial cells for use in liquid composition according to claim 1.

15. Encapsulated viable Gram positive or/and viable Gram negative bacterial cells for use according to claim 14, wherein said bacteria are B. coagulans.

16. Liquid composition according to claim 2, wherein encapsulated viable Gram positive or/and viable Gram negative bacterial cells count is from 1 to 1*10⁹ per polymer particle, from 1 to 1*10¹² of viable cells per gram of capsules wet weight.

17. Liquid composition according to claim 2, wherein said thickeners are selected from Xanthan gum and Celluloses in combination or separately.

18. Liquid composition according to claim 3, wherein said thickeners are selected from Xanthan gum and Celluloses in combination or separately.

19. Liquid composition according to claim 2, wherein said polymer comprises alginate.

20. Liquid composition according to claim 3, wherein said polymer comprises alginate.
